(19) United States
(12) Patent Application Publication
(10) Pub. No.: US 2001/0053547 A1
(43) Pub. Date: Dec. 20, 2001

(54) Method for preparing a platelet composition

(76) Inventor: Sherrill J. Slichter, Vashon, WA (US)

Correspondence Address: CESARI AND MCKENNA, LLP, 88 BLACK FALCON AVENUE, BOSTON, MA, 02210, US

(21) Appl. No.: 09/900,642

(22) Filed: Jul. 6, 2001

Related U.S. Application Data
(62) Division of application No. 09/255,463, filed on Feb. 22, 1999.
Application No. 09/255,463 is a continuation-in-part of application No. 08/566,847, filed on Dec. 4, 1995, now abandoned.

(51) Int. Cl. C12N 5/08

(52) U.S. Cl. 435/372; 435/2

(57) ABSTRACT

Non-immunogenic and/or toleragenic platelet compositions, the compositions being substantially free of white blood cells and/or having the residual white blood cells inactivated are provided. Methods for preparing such compositions using various combinations of filtration, centrifugation and UV irradiation are also provided.

This application is a Divisional of Serial No. 09/255,463, filed Feb. 22, 1999, now ______which is a Continuation-In-Part of Ser. No. 08/566,847, filed Dec. 4, 1995, now abandoned.

### TECHNICAL FIELD

The present invention relates generally to blood products, and more specifically to non-immunogenic and toleragenic platelet compositions, and methods of producing the same.

### BACKGROUND OF THE INVENTION

Patients with a variety of disorders receive intermittent or chronic transfusion support or require tissue grafting to replace a defective organ. For individuals requiring transfusion support, they have either a genetic or acquired deficiency of one or more blood components that require replacement therapy. Many different products prepared from blood are available for transfusion, including both cellular and plasma components. However, repeated exposure to blood products often results in recipient recognition of the foreign transfused antigens. For instance, alloimmune platelet refractoriness occurs in up to 30%-60% of patients requiring repeated platelet transfusions. Such immune recognition of the foreign antigens results in a failure to achieve a benefit from the transfusion and in some circumstances may even cause a transfusion reaction with adverse consequences to the recipient.

Several approaches have been used to either prevent or delay alloimmunization. The majority of the techniques involve giving immunosuppressive therapy to the transfusion recipient to prevent recognition of the transfused foreign antigens. Such immunosuppressive therapy is often inadequate to suppress the recognition process resulting in alloimmunization in spite of the treatment. Furthermore, the immunosuppressive therapy may have undesirable side effects including organ toxicity and immunosuppression of desirable responses such as recognition and destruction of pathogenic bacteria.

Once an immune response to foreign antigens has occurred, there is little evidence that any immunosuppressive therapy is beneficial. Continued adequate transfusion support is possible only if antigen matching between donor and recipient is achieved. Often a matched donor is not available or for some transfusion products so little is known about the antigen systems involved in the immune response that laboratory methods are not available to appropriately select a matched donor.

An alternative approach to preventing alloimmunization, other than immunosuppressing the recipient, is to reduce the immunogenicity of the transfused product. As all transfused blood products are immunogenic and will eventually induce an immune response in most transfused recipients, any procedure that can prevent or at least delay immunization is beneficial. Selecting only antigen compatible donors beginning with the first transfusion is possible in some circumstances, but for the majority of patients not enough donors are available to continue this process or a matching procedure does not exist.

For organ grafting, because there is persistent exposure to foreign tissue antigens, eventual rejection of the grafted tissue occurs. To prevent graft rejection several approaches have been used: recipient immunosuppression, matching tissue antigens of donor and recipient, reducing the immunogenicity of the grafted tissue, or inducing a state of tolerance in the recipient to the foreign antigens of the graft. Furthermore, depending on the tissue being grafted different approaches may be required to achieve a successful graft and combined therapies may be additive in their beneficial effects. For example, in bone marrow transplantation massive doses of chemo-radiotherapy are given to the recipient to destroy the recipient's autologous marrow and to induce immunosuppression to allow engraftment of the donor marrow. Even better results are obtained if marrow donor and recipient are related and well-matched for the major histocompatibility antigen system (HLA). Although post-marrow grafting immunosuppression is usually given, it is for only a limited time.

In contrast, for kidney grafting lesser degrees of immunosuppressive therapy are required to avoid unacceptable marrow and gastrointestinal toxicity. Furthermore, continuous post-grafting immunosuppression is required. Often a related kidney donor is not available, and lesser degrees of HLA matching between donor and recipient are more often accepted than for bone marrow transplantation.

Another major difference between these two types of tissue grafting are the effects of prior transfusions on engraftment. For kidney graft recipients, prior transfusions, particularly from the intended kidney donor, are beneficial apparently by inducing some degree of tolerance to the subsequent kidney graft. However, prior blood transfusions before marrow grafting, especially if the blood has come from the intended marrow donor, markedly increases the risk of graft rejection. Thus, although there are similarities in procedures to enhance organ grafts (immunosuppression and donor-recipient HLA matching) there are clear differences in (1) the amounts, type, and duration of immunosuppression required; (2) the acceptance of non-HLA identity between organ donor and recipient; and, (3) the effects of prior transfusions on enhancing or impairing a subsequent organ graft. Furthermore, even the best combined therapies are not always successful in ensuring a successful organ graft, and there may be substantial toxicities associated with the therapies being used.

Besides using HLA matching and recipient immunosuppression, efforts to directly reduce immunogenicity of the engrafted tissue or to induce tolerance in the recipient, other than by prior blood transfusions in kidney recipients, have been limited. In bone marrow transplantation efforts to purify or enrich the marrow graft for stem cells and eliminate T-lymphocytes that may be responsible for graft vs. host disease (a post-grafting complication) have often resulted in a transplanted marrow that has failed to engraft. Some investigators have stored or cultured the graft in vitro prior to transplantation (skin grafting) to facilitate engraftnent. Most of these latter methods to enhance organ grafting have had limited success.

It would be advantageous to avoid immune recognition by the recipient of incompatible donor antigens and the consequent destruction of allogeneic tissue following transfusion or transplantation.

### SUMMARY OF THE INVENTION

The present invention provides non-immunogenic and/or toleragenic platelet compositions, the compositions being substantially free of white blood cells and/or treated with UV irradiation.

In a related aspect of the present invention, methods of preparing such non-immunogenic and toleragenic platelet compositions are provided, comprising centrifuging a platelet-rich blood product and recovering the supernatant, the supernatant being substantially free of white blood cells; and subsequently filtering the supernatant and recovering the filtrate to produce the platelet composition. Alternatively, the step of filtration may be performed prior to the step of centrifugation.

In another aspect of the present invention, a non-immunogenic platelet composition is provided. The composition may be produced by either (a) centrifuging a platelet-rich blood product, recovering the supernatant and then exposing the composition to UV irradiation; or by (b) filtering the platelet-rich blood product, recovering the filtrate and then exposing the filtrate to UV irradiation. Any of UV-A, UV-B or UV-C irradiation may be used, although UV-B is preferred. It will be evident that the steps of centrifugation, filtration or UV irradiation may be performed in any order.

In any of the methods described above, suitable platelet-rich blood products include platelet-rich plasma, platelet concentrates, apheresis platelets and buffy-coat prepared platelets.

In another aspect of the present invention, a method of inducing immunologic tolerance in a patient to transfused or transplanted tissue is provided. The method generally comprises administering to a patient a non-immunogenic and toleragenic platelet composition, the composition being either substantially free of white blood cells and/or having the white blood cells inactivated by exposing the platelet-rich blood product to UV irradiation, in an amount sufficient to induce immunologic tolerance to subsequently transfused or transplanted tissue. In various embodiments, the transfused or transplanted tissue may be derived from bone marrow, blood, skin, pancreas, bone, liver, heart, lung, kidney or cornea. Within preferred embodiments, the transfused or transplanted tissue is blood.

In a further aspect of the invention, a platelet composition is prepared from a platelet-rich blood product that is both filtered and centrifuged in order to remove residual white blood cells. The combination of filtration with centrifugation removes those white blood cells that are filter adherent and those that are dense, but not necessarily filter adherent, respectively. Preferably, the centrifugation is at approximately 180 times the force of gravity (i.e., 180 xg) for approximately five minutes. In this embodiment, it is not necessary to render the platelet composition substantially free of red blood cells or plasma. Administration of such filtered and centrifuged platelet compositions, moreover, are shown to result in a significantly reduced alloimmune response.

In another aspect of the invention, the platelet compositions that include a filtration step preferably utilize filters that are configured to reduce at least the residual lymphocyte type of white blood cells and also to substantially reduce the residual monocyte type of white blood cells from the platelet-rich blood product.

These and other aspects will become apparent upon reference to the following detailed description.

### DETAILED DESCRIPTION OF THE INVENTION

Prior to setting forth the invention, it may be helpful to the understanding thereof to set forth definitions of certain terms to be used hereinafter.

“Non-immunogenic” as used herein refers to a characteristic of compositions, which when administered to a human, are not associated with either the development of a humoral (antibody) or cellular immune response.

“Toleragenic” as used herein refers to the capacity of a composition to generate an immunologic response consisting of specific non-reactivity of the immune system to a given antigen that, in other circumstances, can induce cell-mediated or humoral immunity.

“Substantially free” as used herein refers to a composition that contains at least a two-three log reduction in white blood cells from baseline, preferably 10⁵ or less (as determined by manual counting using a Neubauer chamber with propidium-iodide staining).

As noted above, the present invention provides non-immunogenic and/or toleragenic platelet compositions, the compositions being substantially free of white blood cells. Critical to achieving such compositions is the use of a combination of filtration and centrifugation steps. As discussed below, such steps may be performed in either order, with equivalent results.

In addition, as noted above, the present invention provides non-immunogenic and/or toleragenic platelet compositions. Such compositions are produced through either a combination of centrifugation and UV irradiation, or filtration and UV irradiation.

Within the context of the present invention, one can utilize any platelet-rich product, such as platelet-rich plasma, platelet concentrates, apheresis platelets or buffy-coat prepared platelet concentrates.

The filtration step(s) as described herein may be performed using a variety of apparatus and procedures. The purpose of filtration is to reduce the number of white blood cells.

Within the subject invention, suitable apparatus for filtration include Pall filters (Pall Biomedical Products, East Hills, N.Y.), such as PLF-1, PL-1, PXL, PLF-10, PL-50 and PL-100, platelet filter TF*IG500 (Terumo, Somerset, N.J.) and platelet filter 4C2477 (Fenwal/Baxter, Chicago, Ill.), with the PXL being preferred. These filters are known as platelet filters, meaning they allow platelets to pass through the corresponding filter medium while trapping primarily white blood cells. White blood cells, moreover, generally comprises three different cell types or populations: lymphocytes, monocytes and granulocytes. The Pall PL series of filters identified above have been shown to be highly effective at significantly reducing the monocyte and granulocyte populations from platelet compositions as compared to other filters. In particular, the preferred filters for use with the present invention are capable of reducing at least the lymphocyte type of white blood cells (e.g., on the order of 1×10⁶ or less per transfusion event) and also to substantially reduce the monocyte type of white blood cells (e.g., on the order of 3×10³ or less per transfusion event). Preferably, the filters reduce the number of detectable monocytes to 2 or less per μL, as described in Avoiding Transfusion Complications: Reducing Costly Complications Associated With Platelet Transfusions from Pall Corp. (citing S. Sowemimo-Coker, A. Kim, E. Tribble, H. Brandwein, B. Wenz White Cell Subsets In Apheresis And Filtered Platelet Concentrates Vol. 38 Transfusion (July 1998), which are both hereby incorporated by reference in their entirety.

In various embodiments, it is the platelet-rich blood product that is passed through the filtration apparatus. However, if the centrifugation step is performed prior to filtration, the collected supernatant is passed through the filtration apparatus. Within the various embodiments, the step of filtration may be performed a second time, with an expectation of a slightly greater degree of leuko-reduction. However, to achieve the advantages described herein, it is not necessary to filter the product more than once.

The step of centrifugation as described herein is preferably performed using a swinging bucket rotor centrifuge (IEC, Needham Heights, Mass.) at approximately 180 xg for 5 minutes. The purpose of this step is to primarily reduce the number of white blood cells that would otherwise not be removed by filtration. Given this purpose, it will be evident that a number of other apparatus may be used. It should also be understood that the centrifugation step may also reduce the number of red blood cells. However, as discussed below, the existence of residual red blood cells in the platelet compositions of the present invention is less relevant.

In various embodiments, it is the platelet-rich blood product that is exposed to centrifugation. However, if the centrifugation step is performed subsequent to filtration, it is the filtrate that is centrifuged, and the resulting supernatant recovered.

In certain embodiments, the centrifuged or filtered platelet composition is further treated by exposure to UV irradiation or UV irradiation can be used alone. Although any of UV-A, UV-B or UV-C irradiation may be used, UV-B is preferred. UV-A represents a wavelength of 320-400 mn, UV-B represents a wavelength of 290-320 nm, and UV-C represents a wavelength of 200-290 nm. The total dose of UV-B irradiation generally results in an exposure of between approximately 600 and 1632 mJ/cm^{2,} with approximately 612 mJ/cm² being preferred. The total dose of UV-C irradiation is preferably between 12 and 36 mJ/cm². The intensity of the irradiation (and therefore a determination of the total exposure) may be obtained through use of a black ray short-wave UV meter (U.V. Products).

The UV-A, UV-B or UV-C irradiation may be performed at any time in the preparation of the platelet composition. It will be evident that a wide variety of apparatus may be used to provide the UV exposure described herein. Such apparatus include a germicidal lamp (General Electric) and a Haemonetics (Braintree, Mass.) UV-B irradiation device.

Upon preparation of the UV-irradiated platelet composition, one can confirm its non-immunogenic and toleragenic properties in vitro by performing mixed lymphocyte culture (MLC) experiments and determining that the residual lymphocytes are no longer able to stimulate or respond in MLC.

As noted above, the non-immunogenic and toleragenic platelet compositions described herein may be used for tolerance induction. Methods of administration of the platelet compositions for this purpose will be evident to those skilled in the art. Tolerance is induced by administering transfusions, generally repeated transfusions, of the platelet composition to a recipient. Transfusions are usually given on a weekly basis, but other schedules are suitable. Generally, from one to eight transfusions are provided, with at least three transfusions being preferred.

To determine whether alloimmune platelet refractoriness has occurred, both platelet responses and antibody measurements are useful. Platelet responses are measured by determining pre-and post-transfusion platelet counts and calculating platelet increments, % platelet recovery, or corrected count increments. Platelet responses can also be determined by radiolabeling the platelets prior to transfusion with a radioactive isotope, such as ⁵¹Chromium or ¹¹¹Indium, and monitoring the disappearance of the radiolabeled platelets from the recipient's circulation. Antibody measurements are made using a variety of tests including platelet and/or lymphocytotoxic antibody tests. Such tests are well known to those skilled in the art.

Alternatively, to determine whether only alloimmunization has occurred, antibody measurements are used. Antibody measurements are made using a variety of tests including platelet and/or lymphocytotoxic antibody tests.

The following examples are offered by way of illustration and not by way of limitation.

### EXAMPLE I

Twenty-seven milliliters of dog blood is drawn via a jugular vein puncture with an 18 G 1 ½″ needle (Becton-Dickinson Immunocytometry Systems, San Jose, Calif.) into a 30 ml syringe (Becton-Dickinson) containing 3 ml of Acid Citrate Dextrose formula A (ACD-A anti-coagulant solution). The blood is then mixed with the ACD-A (Fenwal/Baxter, Chicago, Ill.) by inverting the syringe three times. The anti-coagulated blood is expressed into a 150 ml transfer pack (Baxter) to which 30 ml of Ringer's Citrate Dextrose (RCD) has been added. RCD is prepared by adding 20 ml of 0.2 micron filtered (Gelman Sciences, Ann Arbor, Mich) 1 M sodium citrate (Mallinckrodt, Inc., Paris, Ky) in sterile, non-pyrogenic, injectable water (Baxter) to a 500 ml bag of 5% Dextrose in Ringer's Injection solution (Baxter). The 150 ml pack is inverted three times to mix the ACD blood and RCD and placed in a swinging bucket rotor centrifuge (IEC) for 10 minutes at 240 xg. Platelet rich plasma (PRP) is expressed along with the buffy coat down to the red cell layer via a plasma press (Fenwal) into a second 150 ml transfer pack. The PRP is filtered via gravity flow through a PLF- 1 filter (Pall) connected to a third 150 ml transfer pack. The third transfer pack containing the leucopoor PRP cells is centrifuged in a swinging bucket rotor centrifuge at 1000 xg for 10 minutes. Plasma/RCD is expressed into a fourth 150 ml transfer pack via a plasma press and saved. The remaining cell pellet is gently massaged in the third transfer pack to resuspend it in the residual solution left by the plasma press. Three hundred uCi of sterile, liquid sodium chromate (Amersham), i.e., radioactive Chlromium (⁵¹Cr), is added and the bag is gently inverted five times to mix the solution. The bag is left to incubate at room temperature for 60-90 minutes. The unbound radioactive Chromium (⁵¹Cr) is washed away by transferring the set aside plasma/RCD in the 150 ml transfer pack into the 150 ml transfer pack containing the radioactive Chromium (⁵¹Cr) labeled platelets. The components are mixed by inverting three times, and the radioactive platelets pelleted by spinning for 10 minutes at 1000 xg in a swinging bucket rotor centrifuge. The radioactive plasma is decanted and 5 ½ ml of RCD added to the 150 ml pack. The pelleted platelets are resuspend by gently massaging, decanted into a 13 ml sterile snap cap tube and centrifuged in a swinging bucket for five minutes at 180 xg. The supernatant is aspirated to within 5 mm of the red blood cell (RBC)/white blood cell (WBC) pellet into a 10 ml syringe using a 10 cm long piece of transfer pack tubing attached to the end. Five milliliters is used for injection.

This method of preparation was used to prepare the control platelets (i.e., without filtration or UV irradiation, but leuko-reduced by centrifugation) (row 1, Table 2), the leuko-reduced composition by centrifugation and UV irradiation (with the UV irradiation performed as described in Example II) (row 4, Table 2), and the composition leuko-reduced by filtration (with the filtration performed as described in Example II) and leukocyte-reduced by centrifugation (row 5, Table 2).

### EXAMPLE II

Twenty-seven milliliters of dog blood is drawn via a jugular vein puncture with an 18 G 1 /2″ needle (Becton-Dickinson) into a 30 ml syringe (Becton Dickinson) containing 3 ml of Acid Citrate Dextrose formula A (ACD-A anti-coagulant solution). The blood is then mixed with the ACD-A (Fenwal/Baxter) by inverting the syringe three times. The anti-coagulated blood is expressed into a 150 ml transfer pack (Baxter) to which 30 ml of Ringer's Citrate Dextrose (RCD) has been added. RCD is prepared by adding 20 ml of 0.2 micron filtered (Gelman) 1 M sodium citrate (Mallinckrodt) in sterile, non-pyrogenic, injectable water (Baxter) to a 500 ml bag of 5% Dextrose in Ringer's Injection solution (Baxter). The 150 ml pack is inverted three times to mix the ACD blood and RCD and placed in a swinging bucket rotor centrifuge (IEC) for 10 minutes at 240 xg. Platelet rich plasma (PRP) is expressed along with the buffy coat down to the red cell layer via a plasma press (Fenwal) into a second 150 ml transfer pack. To prepare a filtered leuko-reduced platelet composition, the PRP is filtered via gravity flow through a PLF-1 filter (Pall) connected to another 150 ml transfer pack. To prepare a UV-irradiated platelet composition, the PRP is transferred to a 300 ml UV transmissible bag (Stericell by Terumo, Somerset, N.J.) and illuminated with a monitored dose (International Light Inc., Newburyport, Me.) of 612 mJ/cm² UV-B (Light Sources) with agitation on a Haemonetics Platelet Treatment System (Braintree, MA). To prepare a combined filtered leuko-reduced and UV-B irradiated platelet composition, the PRP is subjected to both procedures in any order. The filtered leuko-reduced PRP, UV-B irradiated PRP or the combined filtered leuko-reduced and UV-B irradiated PRP is transferred to a 150 ml pack in which the cells are pelleted in a swinging bucket rotor centrifuge at 1000 xg for 10 minutes. Plasma/RCD is expressed into a 150 ml transfer pack via a plasma press and saved for later. The remaining cell pellet is gently massaged in the transfer pack to resuspend it in the residual solution left by the plasma press. The platelet suspension is decanted into a 13 ml sterile snap cap centrifuge tube (Falcon) and two sequential 3 ml rinses of the pack with RCD in a 3 ml syringe (Becton-Dickinson) are added to the snap cap tube. This tube is spun in a swinging bucket (IEC) at 180 xg for five minutes.

The PRP is removed with a transfer pipette (Globe Scientific, Inc., Paramus, N.J.) to within 5 mm of the RBC/WBC pellet into another snap cap tube, while the tube containing the RBC/WBC pellet is saved. Three hundred uCi of sterile, liquid sodium 51 chromate (Amersham Corp., Arlington Heights, Ill.) is added and gently vortexed for one second. The tube is left to incubate at room temperature for 60-90 minutes. The unbound radioactive Chromium (⁵Cr) is washed away by transferring the ⁵¹CR/PRP back to the 150 ml pack containing plasma/RCD. The components are mixed by inverting three times, and the radioactive platelets pelleted by spinning for 10 minutes at 1000 xg. in a swinging bucket rotor centrifuge. The radioactive plasma is decanted and 5 ½ ml of RCD added to the 150 ml pack. The pelleted platelets are resuspended by gently massaging, decanted into a 13 ml sterile snap cap tube and centrifuged in a swinging bucket for five minutes at 180 xg. Using a transfer pipette, the platelets are transferred into the saved 13 ml snap cap tube containing the pelleted RBC/WBC. The platelets and RBC/WBC are mixed gently by squeezing and releasing the bulb of the transfer pipette 5 to 10 times and aspirated into a 10 ml syringe using a 10 cm long piece of transfer pack tubing attached to the end. Five milliliters is used for injection.

This method of preparation was used to prepare the leuko-reduced composition prepared by filtration (row 2, Table 2) and the composition prepared by filtration and UV irradiation (row 3, Table 2).

### EXAMPLE III

Pairs of dogs were selected as donor and recipient. Each recipient's response to transfused radiolabeled donor platelets was determined according to a specified transfusion program. In order to detect immunization to the primary donor, up to 8 weeks of treated platelet transfusions were given form the primary donor (1°). In order to evaluate tolerance induction to the primary donor, up to 8 additional weeks of control platelet transfusions from the primary donor were given. In order to evaluate tolerance induction to other donors, up to 8 weeks of control platelet transfusions (centrifuge leuko-reduced) were given from a secondary (2°) and a tertiary donor (3°). For each of these transfusion schedules, ⁵¹Chromium radiolabeled donor platelets were given every week for up to 8 weeks or until the recipient became refractory, defined as less than 5% recovery of donor platelets in the transfused recipient at 24 hours post-injection on 2 sequential occasions. At time of refractoriness, transfusions from the next donor were initiated. In addition to determining platelet refractoriness by radiolabeled platelet survival measurements, some of the transfused recipients also had measurements performed for recipient antibodies against donor platelets, lymphocytes, and red cells by flow cytometry techniques using a FACScan (Becton Dickinson Immunocytometry Systems, San Jose, Calif.).

Table 1 depicts the results of the cell counts done on the platelet products prior to injection as prepared by the techniques outlined under Examples I and II. The abbreviations used in Table 1 (and Table 2 discussed below) are as follows:

| | |
|---|---|
| LR by CENT | leukocyte reduced by centrifugation |
| LR by Fx1 | leukocyte reduced by filtration once |
| LR by Fx2 | leukocyte reduced by filtration twice |
| LR by Fx1 & UVI | leukocyte reduced by filtration once |
| | and UV irradiated |
| LR by Fx1 plus LR by CENT | leukocyte reduced by filtration once |
| | and leukocyte reduced by centrifugation |
| PLAT | platelets |
| WBC | white blood cells |

TABLE 1
RANGE OF TOTAL CELL COUNTS OF TRANSFUSION PRODUCTS

| | Control | | | | | |
|---|---|---|---|---|---|---|
| | Platelets | TREATED PLATELETS | | | | |
| | LR by CENT | LR by FLx1 | LR by Fx2 | LR by Fx1 & UVI | LR by Fx1 plus LR by CENT | LR by Fx1/Buffy Coat |
| PLAT* | 1.2-3.4 × 10⁹ | 1.8-4.7 × 10⁹ | 1.0-3.0 × 10⁹ | 1.20-2.2 × 10⁹ | 0.8-4.0 × 10⁹ | 0.2-2.7 × 10⁹ |
| WBC** | 0-10⁴*** | 0-10⁵ | 0*** | 0-10⁵ | 0*** | 0-3.1 × 10⁴ |

*Determined by automated cell counter (Coulter).
**Determined by manual counting (Neubauer chamber with Propidium-Iodide staining).
***0 Indicates that no cells were seen in the chamber.

Table 2 depicts the results of the transfusion experiments. Using modified platelet transfusions from the primary donor, neither the control platelet products that were white blood cell reduced by centrifugation nor the filtered products—whether filtration was performed either once or twice- were able to prevent platelet alloimmunization, compared to the products that received more than one type of modification; i.e., platelets that were white blood cell reduced by centrifugation or filtration and then UV irradiated or that were white blood cell reduced by both filtration and centrifugation. Within the group of doubly-modified products, none of these products were significantly different from each other in their ability to prevent primary alloimmunization, and they were equally efficacious in maintaining tolerance to control platelet products (centrifuged leuko-reduced) from their primary donors. However, only the platelet products that were white cell reduced by both centrifugation and filtration were able to induce tolerance to control platelet transfusions (centrifuge leuko-reduced) from the 2° and 3° donors to whom they had never previously been exposed (p<0.001, compared to the results of transfusions from 2° and 3° donors given to all other recipients). Due, in part, to the relatively small number of animals in each group, statistical comparisons using binomial distribution (i.e., the p values) were made among the results based on the results from the 2° and 3° donors for the platelet composition that was white blood cell reduced by centrifugation and also further leukocyte-reduced by filtration.

TABLE 2
TRANSFUSION OUTCOMES

| | | | Recipients | Recipients |
|---|---|---|---|---|
| | | | Tolerant To | Tolerant To |
| | Recipient | Recipients Non-Refractory | Non-Rx Platelets*** | Non-Rx Platelets*** |
| Platelets Given | Dogs | To Rx Platelets From 1° Donor | From 1° Donor | From 2° & 3° Donors |
| Control (LR by CENT)* | 21 | 3 (14%) | 1 (5%) | ND |
| LR by Fx1 or x2** | 13 | 4 (31%) | 3 (23%) | 1 (8%) |
| | | p = 0.05 | | |
| LR by Fx1 & UVI | 14 | 10 (71%) p = 0.004 | 8 (51%) | 1 (10%) |
| | | NS | | |
| LR by CENT & UVI | 12 | 7 (58%) | 7 (58%) | 3 (25%) |
| | | NS | | |
| LR by Fx1 plus LR by CENT | 6 | 6 (100%) | 6 (100%) | 6 (100%) |

*Historic controls. No p values are given for comparison with current treated (Rx) arms.
**Of the 8 dogs who received platelets filtered x1, only 3 were non-refractory (38%), and, of the 5 dogs who received platelets filtered x2, only 1 (20%) was non-refractory. Therefore, as there was no difference between the data for filtered x1 and x2, the data have been combined.
***All non-Rx platelets from 1°, 2°, and 3° donors are prepared like the control platelets; i.e., they are LR by CENT.
ND Not done.

### EXAMPLE IV

Table 3 presents the results of additional testing of the platelet compositions described above. Table 3 also presents the results for a platelet composition prepared from a platelet-rich blood product that was subject only to UV-B irradiation. The preparation of such UV-B irradiated platelet compositions is generally described above in connection with Example II without the performance of the filtration step. The corresponding results are shown in lines 3 and 4 of Table 3, using a UV-irradiation device corresponding to the Haemonetics Corp. device utilized in the National Institute of Health's Trial to Reduce Alloimmunization to Platelets (TRAP) study (the initial device) and a Haemonetics Corp. second generation V-irradiation device, respectively.

Table 3 additionally presents the results from further testing of the platelet compositions prepared from the combined steps of filtration and centrifugation, which produced the best results. In particular, tests were performed to evaluate the significance of removing residual RBCs or plasma from this particular platelet composition. In the first test, a plasma composition is formulated from the donor dog and injected into the subject dog along with a radiolabeled, filtered and centrifuged platelet composition. The plasma composition is prepared as follows. Approximately 20 ml of blood is drawn from the donor dog and mixed with an anti-coagulant. The anti-coagulated whole blood is then centrifuged for approximately 10 min. at approximately 1300 xg in a swinging bucket rotor. The plasma layer is removed and gravity filtered through either a PXL or PLF-1 filter from Pall Corp. to generate a leukocyte-reduced plasma composition. Approximately 5 ml of this leuko-reduced plasma composition is added to the filtered and centrifuged radiolabeled leuko-reduced platelet composition and injected into the subject dog. The corresponding results are shown in line 9a of Table 3.

In the second test, a RBC composition is formulated from the donor dog and added back to the radiolabeled, filtered and centrifuged platelet composition prior to injection. The RBC composition is generated as follows. Following centrifugation of anti-coagulated whole blood at 240 xg for 10 min. and removal of the PRP and buffy coat, as described above, the remaining packed RBCs are recovered and re-suspended in RCD. The suspended RBCs are then gravity filtered through two BPF4S red blood cell filters from Pall Corp. to remove leukocytes. The filtered RBCs are then centrifuged at approximately 1300 xg for 10 min. in a swinging bucket rotor. Approximately 2 ml of packed RBCs are recovered and suspended in a 0.9% sodium chloride solution. The recovered packed RBCs are then washed preferably in two cycles with sodium chloride solution. Following each wash cycle, the RBCs are centrifuged at approximately 1100 xg for about 1 minute and the supernatant wash solution is removed. Approximately 1/2 ml of washed, packed RBCs are then added to the radiolabeled, filtered and centrifuged platelet composition and injected into the subject dog. The corresponding results are shown at line 9b of Table 3. As shown, reintroduction of plasma or RBCs had little or no effect on the results obtained from the filtered and centrifuged platelet compositions. Accordingly, it appears to be the removal of leukocytes through the combined filtration and centrifugation steps that produces a substantially non-alloimmune platelet composition and not necessarily the removal of plasma or RBCs.

The results also show that a simple reduction in the overall number of residual leukocytes from the prepared platelet compositions does not necessarily generate an effective composition. In particular, as shown in Table 1 which depicts the results of cell counts in the various platelet compositions described herein, the residual leukocytes remaining in the combined filtered and centrifuged platelet composition are on the same order of magnitude as the residual leukocytes remaining in the filtered once, filtered twice or centrifuged leuko-reduced platelet compositions. Nonetheless, the non-alloimmunization results achieved with the filtered and centrifuged platelet composition are far better than the results from either the filtered or centrifuged leuko-reduced platelet compositions.

TABLE 3
MODIFIED PLATELET TRANSFUSIONS IN THE DOG MODEL

| | A | B | C | D | E |
|---|---|---|---|---|---|
| | Primary Donor | Primary Donor | 2° or 3°* Donors | Primary Donor | 2° or 3° Donors |
| PLATELET MODIFICATION | (Modified Platelets) | (Cent. Leuko-Poor) | (Cent. Leuko-Poor) | (Unmodified) | (Unmodified) |

Single Treatment

| 1) | Centrifugation (Leuko-Reduction) | 3/21 | (14%) | 1/21 | (5%) | ND | | ND | ND |
|---|---|---|---|---|---|---|---|---|---|
| 2) | Filtration (Leuko-Reduction) | 4/13 | (31%) | 3/13 | (23%) | 4/22 | (18%) | ND | ND |

| | | | | | | | | |
|---|---|---|---|---|---|---|---|---|
| 3) | UV-B Irradiated (Initial Device) | 0/9 | (0%) | ND | 3/12 | (25%) | ND | ND |

| | | | | | | | | | |
|---|---|---|---|---|---|---|---|---|---|
| 4) | UV-B Irradiated (Second Generation Device) | 5/11 | (45%) | ND | ND | 3/5 | (60%) | 3/21 | (14%) |

Double Treatment

| 5) | Centrifuged/UV-B Irradiated (Initial Device) | 6/11 | (55%) | 6/11 | (55%) | 6/18 | (33%) | ND | | ND | |
|---|---|---|---|---|---|---|---|---|---|---|---|
| 6) | Filtered/UV-B Irradiated (Initial Device) | 10/14 | (71%) | 9/14 | (64%) | 16/25 | (64%) | 3/5 | (60%) | 2/9 | (22%) |
| 7) | Filtered/UV-B Irradiated | 8/11 | (73%) | ND | | ND | | 5/11 | (45%) | 2/14 | (14%) |
| | (Second Generation Device) | | | | | | | | | | |
| 8) | Filtered/Buffy Coat | 3/5 | (60%) | 3/5 | (60%) | 1/10 | (10%) | 0/1 | | 0/1 | |
| | | 2/3 | (66%) | ND | | ND | | 1/3 | (33%) | 1/6 | (17%) |
| | | 5/8 | (63%) | | | | | | | | |
| 9) | Filtered/Centrifuged | 10/11 | (91%) | 10/11 | (91%) | 17/20 | (85%) | 5/6 | (83%) | 5/11 | (45%) |
| | a) Plus Filtered/Centrifuged Plasma | 4/4 | (100%) | 4/4 | (100%) | 8/8 | (100%) | 3/4 | (75%) | 3/8 | (38%) |
| | b) Plus Filtered/Centrifuged RBC | 4/4 | (100%) | 3/4 | (75%) | 6/8 | (75%) | 2/2 | (100%) | 2/4 | (50%) |
| | Total | 18/19 | (95%) | 17/19 | (89%) | 31/36 | (86%) | 10/12 | (83%) | 10/23 | (43%) |

| | | | | G | | H | |
|---|---|---|---|---|---|---|---|
| | | | | PLATELET TRANSFUSIONS | | | |
| | | | | POST-GRAFTING** | | | |
| | | F | | Primary Donor | | 2° or 3° Donors | |
| PLATELET MODIFICATION | | Skin Graft | | (Unmodified) | | (Unmodified) | |
| Single Treatment | | | | | | | |
| 1) | Centrifugation (Leuko-Reduction) | ND | | ND | | ND | |
| 2) | Filtration (Leuko-Reduction) | ND | | ND | | ND | |
| 3) | UV-B Irradiated (Initial Device) | ND | | ND | | ND | |
| 4) | UV-B Irradiated (Second Generation Device) | ND | | ND | | ND | |
| Double Treatment | | | | | | | |
| 5) | Centrifuged/UV-B Irradiated (Initial Device) | ND | | ND | | ND | |
| 6) | Filtered/UV-B Irradiated (Initial Device) | 0/3 | (0%) | 1/3 | (33%) | 1/4 | (25%) |
| 7) | Filtered/UV-B Irradiated | | | | | | |
| | (Second Generation Device) | | | | | | |
| 8) | Filtered/Buffy Coat | ND | | ND | | ND | |
| | | ND | | ND | | ND | |
| 9) | Filtered/Centrifuged | 2/4 | (50%) | 3/3 | (100%) | 1/4 | (25%) |
| | a) Plus Filtered/Centrifuged Plasma | 1/1 | (100%) | 0/1 | (0%) | 1/1 | (100%) |
| | b) Plus Filtered/Centrifuged RBC | 0/2 | (0%) | 2/2 | (100%) | 0/1 | (0%) |
| | Total | 3/7 | (43%) | 5/6 | (83%) | 2/6 | (33%) |

ND - Not Done.
IP - In Progress
*2° (secondary) or 30° (tertiary) donors.
**A single platelet transfusion was given post-grafting from the donors of the skin grafts to determine if graft rejection caused loss of platelet tolerance.
Data is reported as number of recipient dogs who have not become refractory to 8 weekly platelet transfusions from a donor dog/number of recipient dogs (% non-refractory).

To further explore the effects of another type of combined centrifugation and filtration leuko-reduction process on alloimmunization rates, buffy-coat platelets were used as the starting material. To make buffy-coat platelets, 45 milliliters of dog blood is drawn via ajugular vein puncture with an 18 G 1½″ needle (Becton-Dickinson and Company, Franklin Lakes, N.J.) into a 60 ml syringe (Becton-Dickinson) containing 5 ml of acid citrate dextrose formula A (ACD-A anticoagulant solution; Fenwal/Baxter, Chicago, Ill.). The blood is then mixed with the ACD-A by inverting the syringe six times. The anticoagulated blood is expressed into a 50 ml conical centrifuge tube (Allegiance, McGraw Park, Ill.). The 50 ml tube is placed in a swinging bucket rotor centrifuge (IEC) for 15 minutes at 3800 xg. After centrifugation, the plasma layer and the buffy coat are removed with a transfer pipette (Elkay Products, Inc., Shrewsbury, MA) and placed into a second 50 ml conical tube. This tube is placed on a rotator (Clay Adams, Parisppany, N.J.) and allowed to mix for 20 minutes. The tube is then centrifuged in a swinging bucket rotor for 10 minutes at 480 xg. The supernatant is then removed to a 150 ml transfer pack (Fenwal/Baxter) via a 30 ml syringe which has a cut piece of the 150 ml transfer pack tubing attached to it. The supernatant is then gravity filtered through a PLF-1 or PXL platelet filter (Pall, East Hill, N.J.) attached to a second 150 ml transfer pack. The filtered supernatant (buffy coat prep) is then drawn into a 30 ml syringe (anywhere from 20 to 30 mls). The filtered buffy-coat preparation is then added to radiolabeled, filtered and centrifuged, leuko-reduced platelets as prepared in Table 3, Line 9. The results of these experiments are shown in Table 3, Line 8. Although these buffy coat platelets are a centrifuged and filtered leuko-reduced preparation with relatively low residual white blood cells as illustrated in Table 1, the results show that this preparation is clearly not as effective at preventing platelet refractoriness as the technique used in Table 3, Line 9.

For some of the transfusion experiments reported in Table 3, additional unmodified radiolabeled platelet transfusions from the primary, secondary, and tertiary donors were given to the recipient dogs for up to 8 weeks or until refractoriness developed. These transfusions were given to determine whether tolerance had been induced to platelets that had not been centrifuged leuko-reduced as we had originally used (columns B and C of Table 3). Prior transfusions with the combined centrifuged and filtered leuko-reduced platelet composition (Table 3, Line 9) were able to induce tolerance to unmodified platelets from the primary donor (Table 3, column D, Line 9), and these platelets were still more effective than any other platelet transfusion program in inducing tolerance to unmodified platelets from 2°0 and 3° donors (Table 3, column E, Line 9).

It thus appears that a qualitative difference exists in the residual leukocyte populations of different platelet compositions. In other words, the extent of non-alloimmunization achieved by a particular platelet composition is less a function of the overall leukocyte reduction and more a function of the particular types or populations of residual leukocytes that are removed. That is, the test results appear to demonstrate that certain allostimulatory leukocytes can be removed through filtration, while others must be removed through centrifugation. As described above, there are basically three types of leukocytes: lymphocytes, monocytes and granulocytes. Furthermore, the preferred filters utilized in the present invention are highly efficient at removing not only lymphocyte, but also granulocyte and monocyte types of leukocytes. Accordingly, it is suggested that one of the allostimulatory leukocyte populations is a filter-adherent type of white blood cell (e.g., granulocyte, monocyte or lymphocyte) that is subject to removal through filtration. A likely candidate for the filterable, allostimulatory leukocyte is the monocyte. Nonetheless, a second allostimulatory leukocyte is not filter adherent and thus passes through the filter along with the platelets. These non-adherent leukocytes must be relatively dense and thus subject to removal from the platelet composition through centrifugation. A possible candidate for the dense, allostimulatory leukocyte is a precursor cell to a monocyte or lymphocyte type of white blood cell (such as a dendritic precursor cell). Only with a reduction or removal of both types of allostimulatory leukocyte populations (i.e., both filter-adherent and dense) is a highly non-immunogenic platelet composition formulated.

To determine whether prior transfusions of modified platelet products could induce tolerance not only to platelets but also to subsequent tissue/organ grafts, some of the recipient dogs were given skin grafts from their primary donors following the platelet transfusions given in columns A through E of Table 3. The skin grafts were performed by first anesthetizing the skin graft donor and recipient with sodium pentabarbitol. Then, using sterile surgical technique, a 2 inch square of skin is removed from the donor and two, two-inch squares of skin are removed from the recipient. The grafts to be applied are then scraped clean of any adipose tissue using a scalpel. One of the recipient grafts (auto) and the donor graft (allo) are then placed on each of the two recipient sites with the hair growth of the grafts oriented in the opposite direction of the recipient dog's natural hair growth. The grafts are then sutured into place, and a topical powdered antibiotic (polysporin) is applied. The graft sites are then wrapped in sterile bandages and a cone is placed around the dog's neck to avoid unwanted removal and/or contamination of the bandages. The donor's site is also stitched closed, bandaged, and a cone is placed around the dog's neck. Both donor and recipient dogs are administered an analgesic (Butorfanol) during the recovery period following grafting. Bandages are removed daily, and the sites are inspected, cleaned if necessary, antibiotic is re-applied, and the sites are re-bandaged. At day 11 post-graft, the sutures are removed, and the sites are no longer bandaged. At this time, the dogs are no longer collared, and they are taken off the analgesic.

The graft was considered to have been rejected when, upon a gross examination of the graft site, the graft was deemed “hard” or “gone” or “not viable.” As shown, 3/7 (43%) of the recipients of the centrifuged/filtered leuko-reduced platelets (Table 3, Line 9) did not reject a skin graft from their primary donor (Table 3, column F, Line 9). Furthermore, almost all of the recipient dogs—whether or not they rejected the skin graft from their primary donor—still accepted platelets from their primary donor after grafting (83%) (Table 3, column G, Line 9). This data suggests that prior transfusions with centrifuged/filtered leuko-reduced platelets can induce tolerance to a highly immunogenic graft—such as skin.

Table 4 is a chart summarizing the test protocol utilized to formulate the control platelet compositions and each of the test compositions from platelet rich plasma (the preparation of the buffy coat filtered platelet preparation is described in Example IV). All of the platelet compositions were subjected to the procedures listed in the column marked “Mandatory Steps”, while the modified platelet compositions were subjected to the procedures listed in the column marked “Conditional Steps” and described above in Examples I and II.

TABLE 4
PLATELET COMPOSITION PREPARATION PROTOCOLS

| Mandatory Steps | | Conditional Steps* | |
|---|---|---|---|
| 1. | Draw 30 cc of donor whole blood into a syringe | | |
| | containing 3 cc of ACD. | | |
| 2. | Transfer whole blood to a bag and dilute with | | |
| | RCD (1:1) to improve the separation of PRP | | |
| | from the RBC layer. | | |
| 3. | Soft centrifugation (250 g for 10 min.) | | |
| 4. | Transfer PRP to another bag, including buffy | | |
| | coat layer, to optimize platelet harvesting. | 4a. | Filtration x1 or x2. |
| | | 4b. | UV-B irradiation. |
| 5. | Hard centrifugation (900 g for 10 min.). | | |
| 6. | Transfer supernatant plasma/RCD mixture to | | |
| | satelite bag and retain | | |
| 7. | Re-suspend platelet concentrate in approximately | | |
| | 5 cc of residual plasma/RCD. | 7a. | Soft centrifugation of platelet concentrate |
| | | | at 180 g for 5 min. Draw off supernatant |
| | | | platelets for radiolabeling (Step 8). Retain |
| | | | pellet. |
| 8. | Add 300 μCu ⁶¹Cr to platelet concentrate and | | |
| | incubate for 60 min. at room temperature. | | |
| 9. | Add back autologous plasma/RCD from Step 6. | | |
| 10. | Hard centrifugation (900 g for 10 min.). | | |
| 11. | Discard supernatant plasma/RCD. | | |
| 12. | Resuspend platelets in 6 cc of added RCD. | | |
| 13. | Final soft centrifugation at 180 g for 5 min. | | |
| | Draw off supernatant platelets. | 13a. | Return unlabeled-but treated-pellet |
| | | | from 7a to supernatant platelets. |
| 14. | Retain 1 ml aliquot of supernatant platelets for cell | | |
| | and radioactive counting and transfuse the remain- | | |
| | ing 5 mls. | | |

See text for abbreviations and reagent/supply information.
*These conditional steps are performed-or not-depending on the treatment assignment of the recipient dog.

The foregoing description has been directed to specific embodiments of this invention. It will be apparent, however, that other variations and modifications may be made to the described embodiments with the attainment of some or all of their advantages. Accordingly, this description should be taken only by way of example and not by way of limitation. It is the object of the appended claims to cover all such variations and modifications as come within the true spirit and scope of the invention.

What is claimed is:

1. A method of preparing a toleragenic platelet composition that is substantially free of leukocytes from a platelet rich blood product including red blood cells, platelets, both dense and filter-adherent leukocytes and plasma, said method comprising the steps of
filtering the blood product to remove the filter-adherent leukocytes;
centrifuging the filtered blood product at a first speed to express off plasma and produce a platelet concentrate;
resuspending the platelet concentrate in residual plasma;
subsequently centrifuging the suspension at a second speed less than the first speed to separate a supernatant platelet fraction from said red blood cells and dense leukocytes, and
recovering the supernatant platelet fraction as said toleragenic platelet composition.

2. The method defined in claim 1 wherein the filtering step is repeated at least once.

3. The method defined in claim 1 wherein the filtering step is performed using one of a Pall PL series of white blood cell filters.

4. The method defined in claim 1 wherein the step of subsequently centrifuging is performed at approximately 180× the force of gravity.

5. The method defined in claim 4 wherein the step of subsequently centrifuging is performed for approximately five minutes.

6. The method defined in claim 1 wherein the platelet rich blood product is selected from the group consisting of platelet-rich plasma, platelet concentrate, apheresis platelets and buffy-coat platelet preparations.

7. A method of preparing a toleragenic platelet composition that is substantially free of leukocytes from a platelet rich blood product including red blood cells, platelets, both dense and filter-adherent leukocytes and plasma, said method comprising the steps of centrifuging the blood product at a first speed to express off plasma and produce a platelet concentrate;
resuspending the platelet concentration in residual plasma;
subsequently centrifuging the suspension at a second speed lower than said first speed to separate a supernatant platelet fraction from said red blood cells and dense leukocytes;
filtering the supernatant platelet fraction to remove the filter-adherent leukocytes, and
recovering the filtered supernatant platelet fraction as said toleragenic platelet composition.

8. The method defined in claim 7 wherein the filtering step in repeated at least once.

9. The method defined in claim 7 wherein the filtering step is performed using one of a Pall PL series of white blood cell filters.

10. The method defined in claim 7 wherein the step of subsequently centrifuging is performed at approximately 180× the force of gravity.

11. The method defined in claim 7 wherein the step of subsequently centrifuging is performed for approximately five minutes.

12. The method defined in claim 7 wherein the platelet rich blood product is selected from the group consisting of platelet-rich plasma, platelet concentrate, apheresis platelets and buffy-coat platelet preparations.
